(19) United States
(12) Patent Application Publication
(10) Pub. No.: US 2026/0207226 A1
(43) Pub. Date: Jul. 23, 2026

(54) SURGICAL DISTRACTION DEVICE AND METHODS OF USE TO FACILITATE PERCUTANEOUS REDUCTION AND FIXATION OF CALCANEUS FRACTURES

(71) Applicant: OrthoSmith LLC, Fairhaven, NJ (US)

(72) Inventors: William Vanderveer Probasco, Fairfield, CT (US); Kevin Andrew Schafer, Fairhaven, NJ (US)

(21) Appl. No.: 19/456,309

(22) Filed: Jan. 22, 2026

Related U.S. Application Data
(60) Provisional application No. 63/748,152, filed on Jan. 22, 2025.

(51) Int. Cl. A61B 17/64; A61B 17/56; A61B 17/66

(52) U.S. Cl. CPC A61B 17/6425; A61B 17/66; A61B 2017/564

(57) ABSTRACT

One method, among others, of calcaneus distraction of the present disclosure comprises placing one or more guide pins through an ankle; attaching one or more calcaneus distraction devices to the one or more guide pins; distracting the one or more guide pins using the one or more calcaneus distraction devices; and/or securing reduction of calcaneus fragments through fixation of a distractor fastener, wherein the fixation of the distractor fastener is facilitated by a drill targeting guide.

### CROSS-REFERENCE TO RELATED APPLICATION

This application claims priority to co-pending U.S. provisional application entitled, “Surgical Distraction Device and Methods of Use to Facilitate Percutaneous Reduction and Fixation of Calcaneus Fractures,” having application number 63/748,152, filed Jan. 22, 2025, which is entirely incorporated herein by reference.

### BACKGROUND

Intra-articular calcaneus fractures are one of the most challenging clinical scenarios for an orthopedic surgeon to manage. These injuries often occur as high energy traumas in younger patients that rely on excellent mobility to provide for themselves and their families. Unfortunately, nonoperative care of these injuries often results in suboptimal outcomes. Even after the fractures have healed, most patients will experience regular pain with ambulation, difficulty with shoe wear due to deformed heel, altered gait, physical limitations at work, and will go on to develop post-traumatic arthritis that may require surgical care. While challenging, a successful surgical reduction and fixation of these fractures can improve patient outcomes significantly.

The reduction and fixation of intra-articular calcaneus fractures is one of the most difficult surgical tasks in orthopedic surgery. Each stage of the surgery has its associated challenges. Surgery begins with an exposure of the fracture and subtalar joint, which requires dissection over an anatomic region with a thin soft tissue envelope. This relatively less forgiving soft tissue becomes even more fragile as the underlying bone fractures into multiple pieces, causing significant swelling and skin blistering. As such, many surgeons will wait one to three weeks for swelling to improve before proceeding with surgery. Nevertheless, wound complications are still common, occurring in 9-25% of cases reported in the literature.

After surgical exposure, the reduction of the fracture fragments is the next, and greatest challenge. The posterior facet of the subtalar joint is often in multiple pieces that are impacted and rotated. Next, the tuberosity fragment, or posterior part of the heel bone, must be manipulated in multiple planes to restore the length and proper alignment of the heel bone relative to the foot.

Additionally, in many fractures, the anterior process may be in multiple pieces that must be restored to their proper positions. To make matters even more challenging, some of these fragments must be manipulated simultaneously to achieve an adequate reduction of each individual piece. This often requires multiple skilled hands and surgical reduction tools, and placing temporary fixation devices that must then later be removed. After reducing each fracture fragment, the surgeon then must stabilize the surgical reductions. This can be performed with many different types of surgical implants, dictated by the pattern of the fractures and surgeon preference. This can be particularly challenging in cases of greater comminution and bony impaction. Traditionally, a lateral plate with locking fixation is applied, which requires a sizeable incision. This larger incision not only increases the risk of wound complications, but may also delay postoperative rehabilitation and restoration of motion as the incision is protected until adequate healing.

More recently, studies have demonstrated that isolated screw fixation of calcaneus fractures can yield similar results in comparison to traditional plating techniques. When inserted through percutaneous incisions requiring minimal soft tissue disruption, screw fixation constructs can provide similar biomechanical construct strength, comparable radiographic reductions, and result in similar patient outcomes. Importantly, percutaneous reduction and fixation techniques can be performed immediately after the injury, and allow for more accelerated postoperative rehabilitation given lower concerns for wound healing with early motion.

Despite the published advantages of percutaneous calcaneal fracture reduction and fixation, these techniques are not routinely practiced. These techniques are challenging, and there are few tools available to surgeons that facilitate a percutaneous reduction. As a result, most surgeons resort to the more classic techniques learned during their training and make a formal, larger exposure despite the elevated risk profile.

### SUMMARY

Embodiments of the present disclosure provide novel calcaneus distraction systems and related devices and methods. Accordingly, various embodiments of the present disclosure concern a novel calcaneus distraction device for controlled manipulation and fixation of intra-articular calcaneus fracture fragments. Also included is a novel distractor fastener that is designed to resist fastener backout, and a drill targeting guide to facilitate fastener placement.

In one embodiment, an exemplary calcaneus distraction device is first attached to two guide pins that are first placed through the foot, with these guide pins placed in a consistent orientation relative to the fracture deformities as visualized on X-ray. These guide pins can then be manipulated by the attachment of a novel calcaneus distraction device, which is designed to accommodate this specific fracture deformity pattern. Once in place, the calcaneus distraction device allows the surgeon to move the fracture fragments with control and precision under X-ray guidance, facilitating correction of the fracture deformities without a formal surgical incision. The bone fragments can then be stabilized in a minimally invasive fashion with the calcaneus distraction device still in place, theoretically decreasing the risks of this operation and improving patient outcomes. Novel fasteners, as described herein, enhance the fixation strength of these fractures by resisting bone collapse and fastener back out. A novel drill targeting guide may also be utilized to correctly position the fasteners under the posterior facet of the subtalar joint. Embodiments of the present disclosure enable the surgical reduction and fixation of calcaneus bone fractures easier and more reproducible for surgeons, and most importantly enhance the outcomes of patients with these fractures.

One embodiment of the system, among others, comprises a calcaneus distraction device having: a threaded distractor arm having an L-shape comprising a first elongated longitudinal body extending between a first end and a second end, and a second elongated longitudinal body extending between the second end and a third end, wherein the first elongated longitudinal body and the second elongated longitudinal body are connected at an approximately 90 degree angle to one another, the first end having at least a first ball socket and the second elongated longitudinal body having threads on at least two opposing surfaces of the second elongated longitudinal body, the third end being configured to removably couple with a sliding distractor arm; the sliding distractor arm having a third elongated longitudinal body extending between a fourth end and a fifth end, and a fourth elongated longitudinal body extending between the fifth end and a sliding end, wherein the third elongated longitudinal body and the fourth elongated longitudinal body are connected at an approximately 90 degree angle to one another, the fourth end having at least a second ball socket and the fourth elongated longitudinal body having a channel throughout its longitudinal axis configured to fit the third end of the threaded distractor arm, wherein the third end of the threaded distractor arm is removably coupled to the sliding distractor arm by a distractor nut that is configured to rotate along the threads of the threaded distractor arm to displace the sliding distractor arm along the longitudinal axis of the threaded distractor arm; and/or a plurality of spherical ball guides configured to rotate within the first or second ball sockets, the spherical ball guides having a through channel configured to fit a guide pin.

In one or more aspects of such systems, the spherical ball guides comprise an actuator channel configured to fit a set screw for locking the guide pin into a spherical ball guide at a desired position, wherein the actuator channel is perpendicular to the through channel, the threaded distractor arm and the sliding distractor arm comprise a radiolucent material, the first end further comprises a first fastener channel adjacent to the first ball socket, the first fastener channel extending perpendicularly through the first end and being configured to fit a first fixation fastener, and/or the fourth end further comprises a second fastener channel adjacent to the second ball socket, the second fastener channel extending perpendicularly through the fourth end and being configured to fit a second fixation fastener.

In one or more aspects, such systems may further comprise a distractor fastener having an elongated cylindrical body having a sixth end and a seventh end, wherein an outer diameter tapers along the elongated cylindrical body from the sixth end to the seventh end, the outer diameter of the sixth end being equal to or greater than the outer diameter of the seventh end, and/or wherein a channel having an inner diameter extends axially through the elongated cylindrical body; and/or helical threads traversing along the elongated cylindrical body from the sixth end to the seventh end, wherein the helical threads change a thread profile at a point along the elongated cylindrical body.

In one or more aspects, such system may further comprise a drill targeting guide comprising a semicircular body having an eighth end and a ninth end, the eighth end having a target point extending perpendicularly from the eighth end, the ninth end having a channel extending perpendicularly through the ninth end, wherein the channel is configured to fit a guide wire; and/or a distractor fastener having an elongated cylindrical body having a tenth end and an eleventh end, wherein an outer diameter tapers along the elongated cylindrical body from the tenth end to the eleventh end, the outer diameter of the tenth end being equal to or greater than the outer diameter of the eleventh end, and/or wherein a channel having an inner diameter extends axially through the elongated cylindrical body that is configured to receive the guide wire after being positioning using the drill targeting guide; and/or helical threads traversing along the elongated cylindrical body from the tenth end to the eleventh end, wherein the helical threads change a thread profile at a point along the elongated cylindrical body.

One embodiment of the method, among others placing one or more guide pins through an ankle; attaching one or more calcaneus distraction devices to the one or more guide pins; distracting the one or more guide pins using the one or more calcaneus distraction devices; and/or securing reduction of calcaneus fragments through fixation of a distractor fastener, wherein the fixation of the distractor fastener is facilitated by a drill targeting guide.

In one or more aspects for such methods, the one or more one or more calcaneus distraction devices comprise: a threaded distractor arm having an L-shape comprising a first elongated longitudinal body extending between a first end and a second end, and a second elongated longitudinal body extending between the second end and a third end, wherein the first elongated longitudinal body and the second elongated longitudinal body are connected at an approximately 90 degree angle to one another, the first end having at least a first ball socket and the second elongated longitudinal body having threads on at least two opposing surfaces of the second elongated longitudinal body, the third end being configured to removably couple with a sliding distractor arm; the sliding distractor arm having a third elongated longitudinal body extending between a fourth end and a fifth end, and/or a fourth elongated longitudinal body extending between the fifth end and a sliding end, wherein the third elongated longitudinal body and the fourth elongated longitudinal body are connected at an approximately 90 degree angle to one another, the fourth end having at least a second ball socket and the fourth elongated longitudinal body having a channel throughout its longitudinal axis configured to fit the third end of the threaded distractor arm, wherein the third end of the threaded distractor arm is removably coupled to the sliding distractor arm by a distractor nut that is configured to rotate along the threads of the threaded distractor arm to displace the sliding distractor arm along the longitudinal axis of the threaded distractor arm; and/or a plurality of spherical ball guides configured to rotate within the first or second ball sockets, the spherical ball guides having a through channel configured to fit a guide pin.

In one or more aspects of such methods, the spherical ball guides comprise an actuator channel configured to fit a set screw for locking the guide pin into a spherical ball guide at a desired position, wherein the actuator channel is perpendicular to the through channel.

In one or more aspects of such methods, the distractor fastener comprises: an elongated cylindrical body having a sixth end and a seventh end, wherein an outer diameter tapers along the elongated cylindrical body from the sixth end to the seventh end, the outer diameter of the sixth end being equal to or greater than the outer diameter of the seventh end, and/or wherein a channel having an inner diameter extends axially through the elongated cylindrical body that is configured to receive a guide wire; and/or helical threads traversing along the elongated cylindrical body from the sixth end to the seventh end, wherein the helical threads change a thread profile at a point along the elongated cylindrical body.

In one or more aspects of such methods, the drill targeting guide comprises a semicircular body having an eighth end and a ninth end, the eighth end having a target point extending perpendicularly from the eighth end, the ninth end having a channel extending perpendicularly through the ninth end, wherein the channel is configured to fit a guide wire.

In one or more aspects of such methods, the distractor fastener comprises: an elongated cylindrical body having a tenth end and an eleventh end, wherein an outer diameter tapers along the elongated cylindrical body from the tenth end to the eleventh end, and wherein a channel having an inner diameter extends axially through the elongated cylindrical body that is configured to receive the guide wire after being positioned using the drill targeting guide; and helical threads traversing along the elongated cylindrical body from the tenth end to the eleventh end, wherein the helical threads change a thread profile at a point along the elongated cylindrical body, wherein the outer diameter of the tenth end is equal to or greater than the outer diameter of the eleventh end.

Other systems, methods, features, and advantages of the present disclosure will be or become apparent to one with skill in the art upon examination of the following drawings and detailed description. It is intended that all such additional systems, methods, features, and advantages be included within this description and be within the scope of the present disclosure.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows an assembled calcaneus distraction device in accordance with various embodiments of the present disclosure.

FIG. 2 is an illustration of an exploded view of an exemplary calcaneus distraction device and its components, including a set screw, a torx screw, an associated distractor nut, and distractor arms, in accordance with various embodiments of the present disclosure.

FIG. 3 is an illustration depicting an exemplary distractor arm component that is threaded, in accordance with various embodiments of the present disclosure.

FIG. 4 is an illustration of a slide distractor arm component of an exemplary calcaneus distraction device that telescopes along the threaded distractor arm component of FIG. 3, in accordance with various embodiments of the present disclosure.

FIG. 5 is an illustration of a distractor nut component of the calcaneus distraction device that moves the slide distractor arm component (FIG. 4) along the threaded distractor arm component (FIG. 3) of an exemplary calcaneus distraction device, in accordance with various embodiments of the present disclosure.

FIG. 6 is an illustration of a distractor spherical ball guide that fits in the ball sockets of the threaded distractor arm (FIG. 3) and slide distractor arm components of an exemplary calcaneus distraction device (FIG. 4), in accordance with various embodiments of the present disclosure.

FIG. 7 is an illustration of an exemplary calcaneus distraction device assembled at a minimum length, with the slide distractor arm component fully advanced along the threaded distractor arm component, in accordance with various embodiments of the present disclosure.

FIG. 8 is an illustration of the slide distractor arm component on the threaded distractor arm component in the reverse position in accordance with various embodiments of the present disclosure.

FIG. 9 is an illustration of an exemplary calcaneus distraction device assembled at the maximum length, with the slide distractor arm component placed on the threaded distractor arm component at the maximum allowable distance.

FIG. 10 shows a 3D printed distractor fastener model of a fully threaded tapered screw, with a change in the direction of the thread profile halfway along the screw, in accordance with various embodiments of the present disclosure.

FIG. 11 is an illustration of the dimensions of an exemplary distractor fastener in the form of a fully threaded screw including the taper of the screw head, and a change in the thread profile halfway along the screw length, in accordance with various embodiments of the present disclosure.

FIG. 12 shows a 3D printed model of a drill targeting guide in accordance with various embodiments of the present disclosure.

FIG. 13 is an illustration of an exemplary drill targeting guide and its dimensions in accordance with various embodiments of the present disclosure.

FIG. 14 shows an X-ray image (left) of a lateral image of a calcaneus with two transfixing guide pins positioned using systems and methods of the present disclosure next to a clinical image (right) of the guide pins and the fracture deformity, in accordance with various embodiments of the present disclosure.

FIG. 15 shows a vector of distraction (solid arrow) between the two placed guide pins, allowing for correction of height and length of the calcaneus, using systems and methods of the present disclosure.

FIGS. 16A-16C depicts three axial radiographs of a calcaneus showing the initial shortened and varus position (FIG. 16A), corrected position with distraction along the guide pins (FIG. 16B), and maintained corrected position with fastener fixation (FIG. 16C), in accordance with the present disclosure.

FIGS. 17A and 17B depicts two lateral radiographs of a calcaneus, showing manipulation of the posterior facet osteochondral fragment (black dashed line, FIG. 17A and FIG. 17B) back into the anatomic position (FIG. 17B), in accordance with the present disclosure.

FIG. 18 depicts two lateral radiographs showing a fastener construct stabilizing a reduction of an intra-articular calcaneus fracture, in accordance with the present disclosure.

### DETAILED DESCRIPTION

The present disclosure presents a novel calcaneus distractor device. A novel distractor fastener and drill targeting guide are also discussed herein and are to be used in conjunction with the novel calcaneus distraction device, in accordance with various embodiments of the present disclosure. In the following description, for purposes of explanation, numerous specific details are set forth in order to provide a thorough understanding of the present disclosure. It will be evident, however, to one skilled in the art that various embodiments of the present disclosure may be practiced without these specific details. Accordingly, the present disclosure is to be considered as an exemplification of the invention, and is not intended to limit the invention to specific embodiments illustrated by the figures or description below.

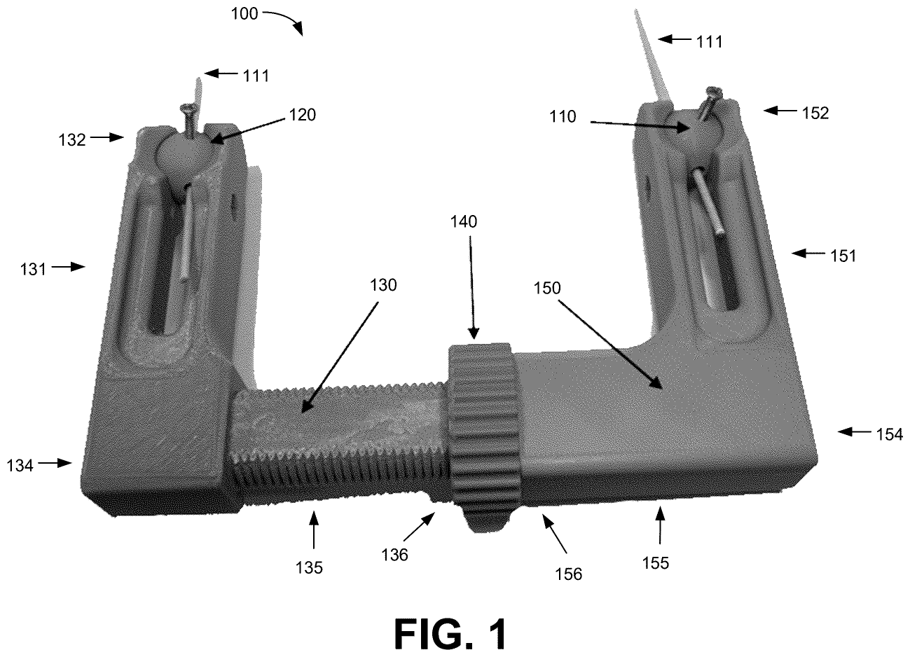

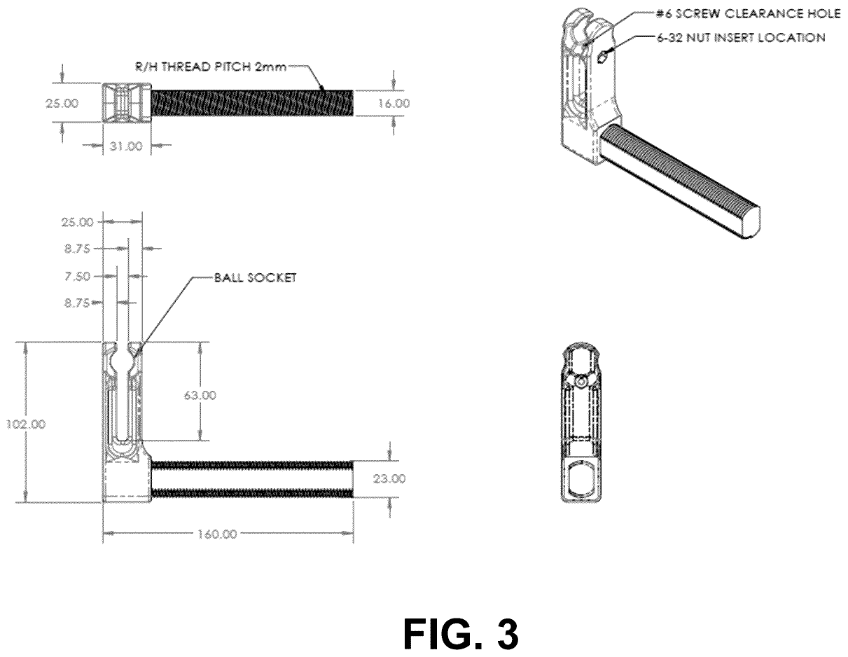

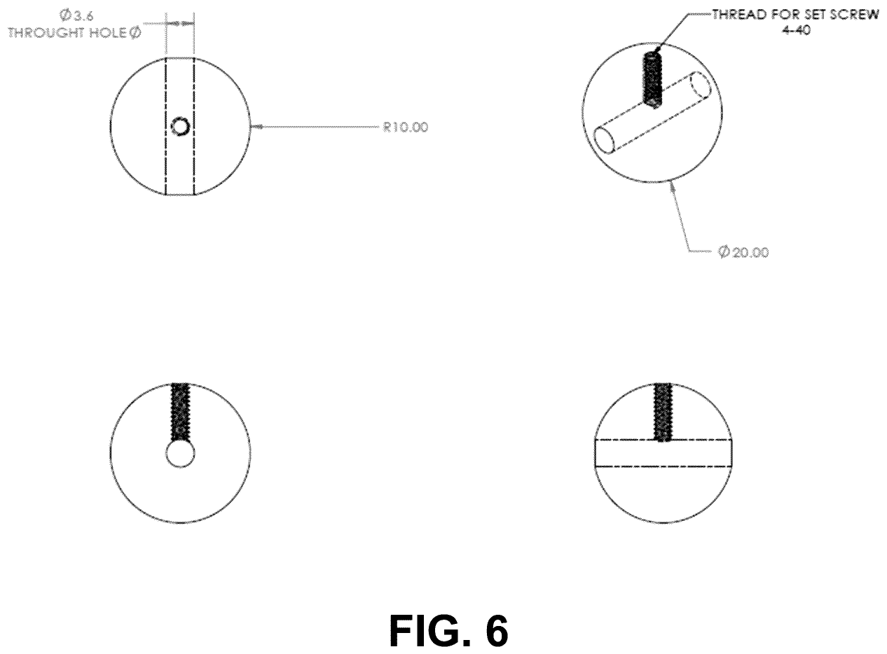

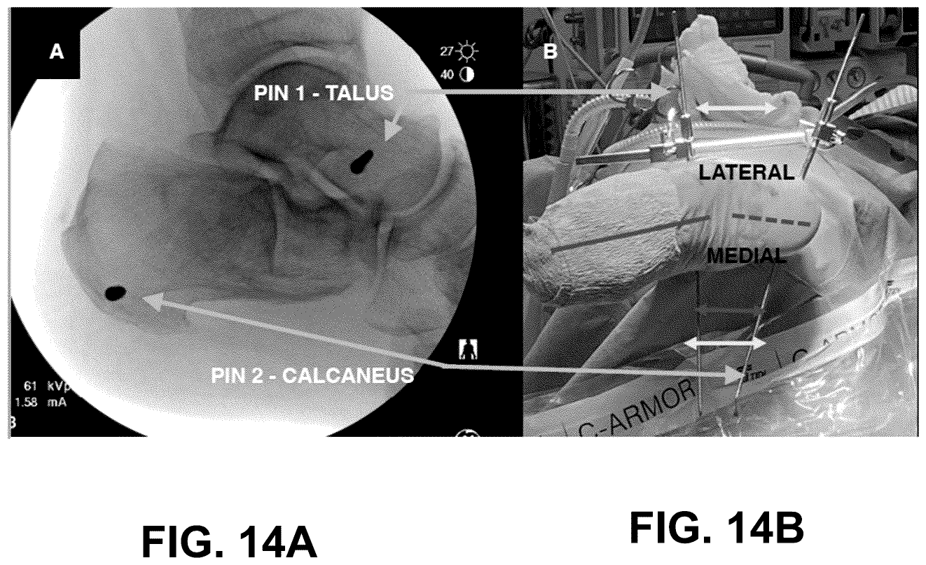

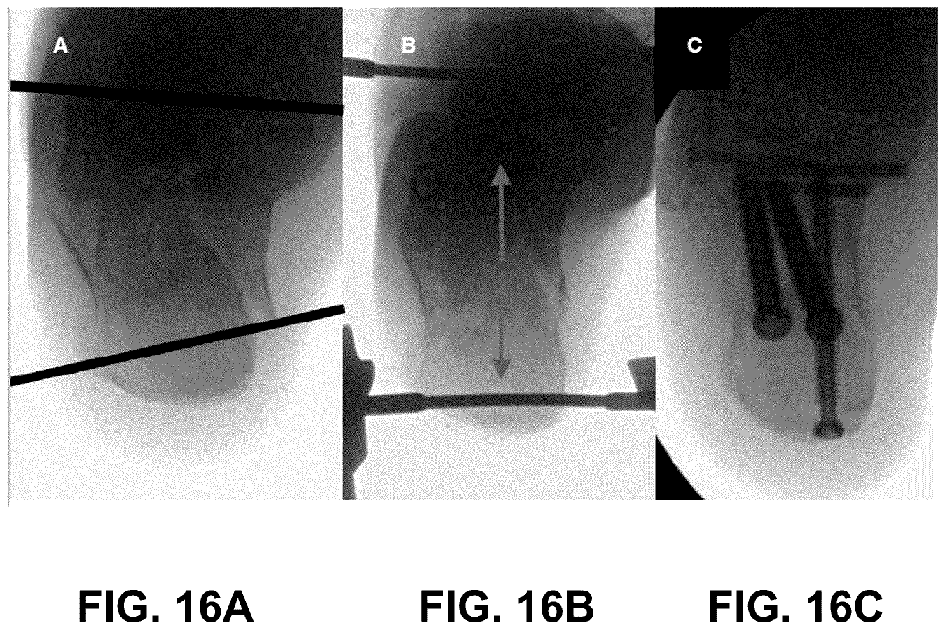

The present disclosure will now be described by referencing the drawings representing certain embodiments. FIG. 1 depicts a 3D printed physical model of a calcaneus distraction device 100, fully assembled. In various embodiments, the novel device 100 may be manufactured from various solid materials, such as hard resins, metals, polymers, etc. In this illustration, wooden dowels are placed through spherical distractor ball guides 110 that rest in each ball socket component 120 (see FIGS. 3, 4, 6) and are meant to represent guide pins or wires 111. Accordingly, during actual use, these spherical distractor ball guides 120 will accommodate metal guide pins or wires 111, such as, but not limited to, having a 3.5 mm diameter (FIG. 6). The spherical ball guides 120 allow for the accommodation of two guide pins (or wires) at drastically different angles. This is necessary as the surgical technique starts by placing two guide pins in significantly divergent paths before bringing them to a relatively parallel position to correct the deformity of the calcaneus fracture fragments (FIGS. 16A-B). The divergent pin placement is pictured in FIG. 14 (right), which is a clinical image of the foot in the operating room, with the patient lying on their side. This image shows the varus deformity of the calcaneus (dashed line) relative to the midfoot and forefoot (solid line). By placing each guide pin perpendicular to its corresponding axial boney alignment under X-ray guidance, the guide pins appear to converge on the medial side of the foot, as opposed to diverge on the lateral side. The corresponding radiographic appearance of these guide pins is depicted in FIG. 14 (left), and FIG. 16A. Here, FIG. 16A best shows the typical fracture pattern in this injury, with the calcaneal tuberosity piece shortened and in a varus posture with the guide pins converging on the medial side of the hindfoot. The novel calcaneus distraction device is then secured to the guide pins and attached to each side of the foot so that a deformity correction can be performed.

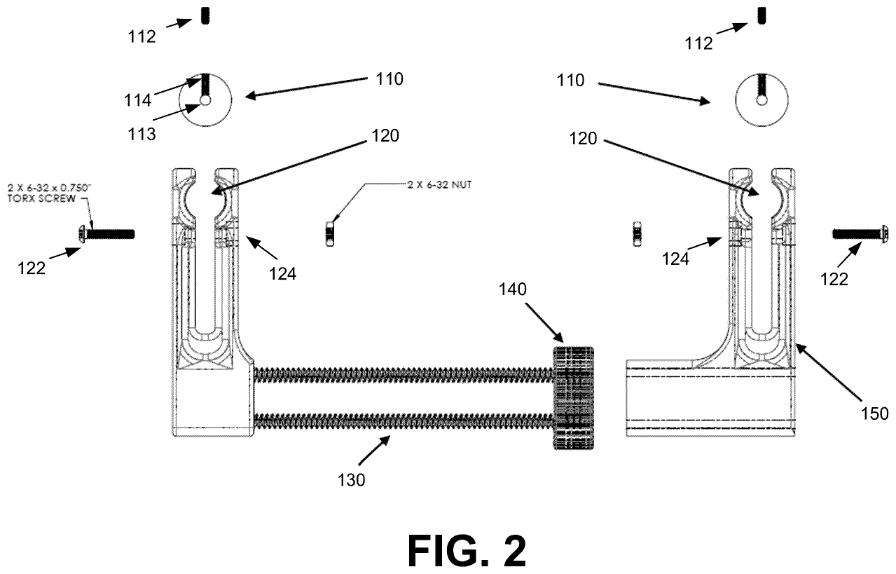

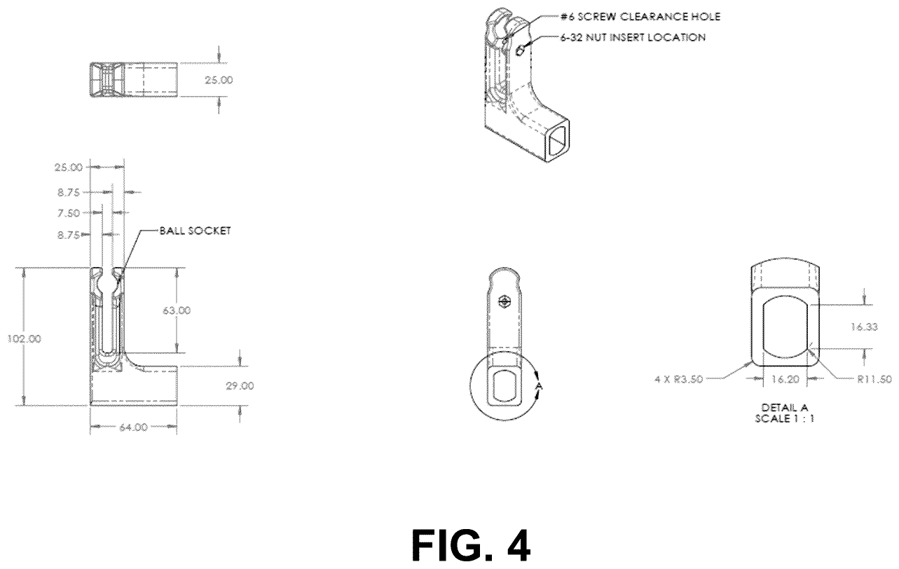

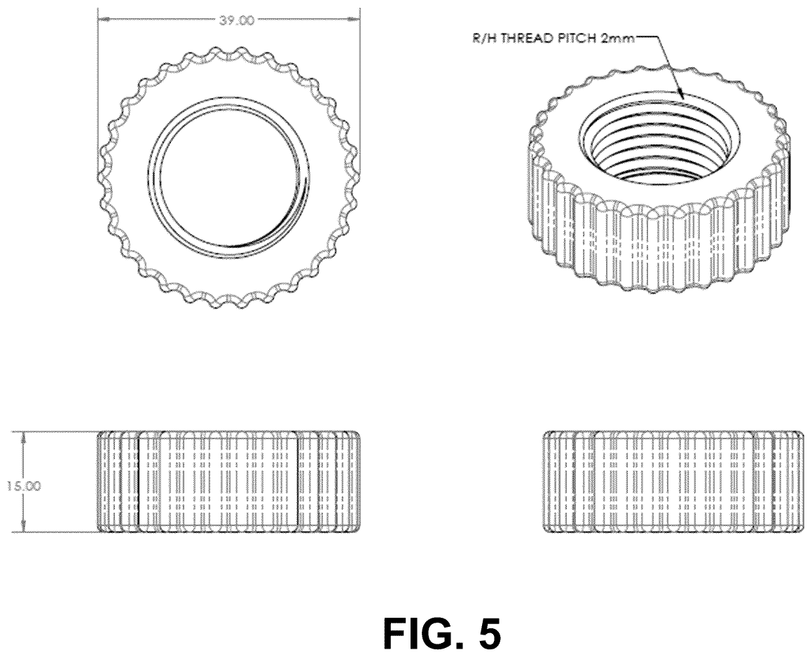

The functionality of the calcaneus distraction device is best understood by an analysis of its subcomponents. FIG. 2 depicts an exploded view of an exemplary calcaneus distractor device, showing a threaded distractor arm component 130 (FIG. 3) with an attached distractor nut component 140 (FIG. 5), and a slide distractor arm component 150 (FIG. 4). In various embodiments, the threaded distractor arm 130 and the sliding distractor arm 150 comprise a radiolucent material.

Also shown are spherical distractor ball guide components 110 having a corresponding set screw 112 that is used to lock the guide pins into the spherical ball guides at the desired position. Once the spherical ball guides 110 are then manipulated into the desired position within ball socket components 120, a fixation fastener 122 (e.g., a torx screw) can then be used to lock the spherical ball guide 110 in the desired position within the respective distractor arm 130, 150 by inserting the fixation fastener 122 in a fixation fastener channel 124 (extending perpendicularly through the arm end) adjacent to the spherical ball guide 110 in a respective distractor arm 130, 150. This novel design allows for the guide pins to be fixed to the spherical ball guides 110 independent of fixation of the spherical ball guides 110 within the corresponding ball sockets 120 of each distractor arm of the calcaneus distraction device. This is essential for this surgical procedure as the spherical ball guides 110 (and well-fixed traversing guide pins) need to be manipulated from a significantly divergent position to a relatively parallel position without constraint from the calcaneus distraction device 100 (FIG. 14 (right)). Once the desired guide pin alignment has been achieved, the torx screw 112 can then be advanced to lock the spherical ball guide position in each distractor arm, holding the reduction while the surgeon proceeds with fixation.

FIG. 3 depicts the threaded distractor arm 130 of an exemplary calcaneus distraction device 100. In one embodiment, the threaded component of the distractor arm has a 2 mm pitch. The dimensions of one embodiment of this component are detailed in the figure, which show that a length of the arm is 160 mm and a height of the arm is 102 mm, in a non-limiting embodiment. Correspondingly, FIG. 4 illustrates the slider distractor arm 150 of the calcaneus distraction device 100, which telescopes along the threaded distractor arm 130 depicted in FIG. 3. This allows for controlled movement of the slider distractor arm 150 and the attached spherical ball guide 110. The dimensions of one embodiment of this component are detailed in the figure, which show that a height of the slider distractor arm 150 to be 102 mm and a width to be 64 mm, in a non-limiting embodiment. FIG. 5 depicts the distractor nut component 140 (of FIG. 2) that allows the slider distractor arm 150 (FIG. 4) to be carefully moved along the threaded distractor arm 130 (FIG. 3). The dimensions of one embodiment of this component are detailed in the figure, which show an outer diameter of the distractor nut to be 39 mm in a non-limiting embodiment.

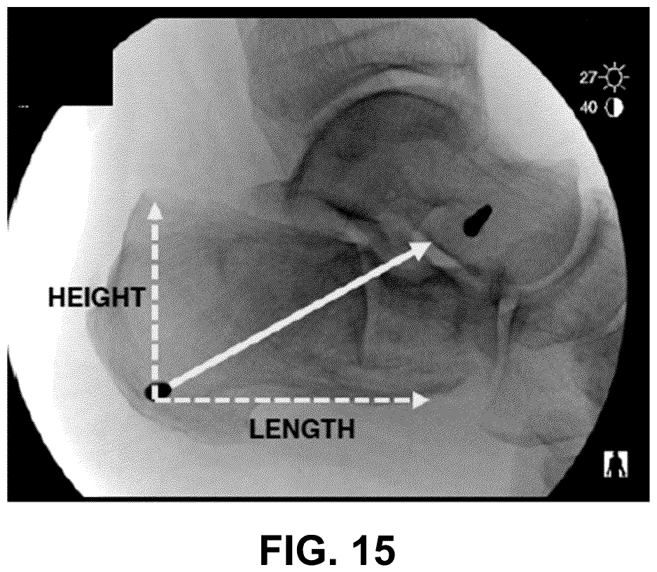

FIG. 15 shows a vector of distraction (solid arrow) between the two placed guide pins, allowing for correction of height and length of the calcaneus, using systems and methods of the present disclosure. The axis of distraction, when the slide arm is lengthened relative to the threaded arm, allows the operator the ability to increase both calcaneal height and length.

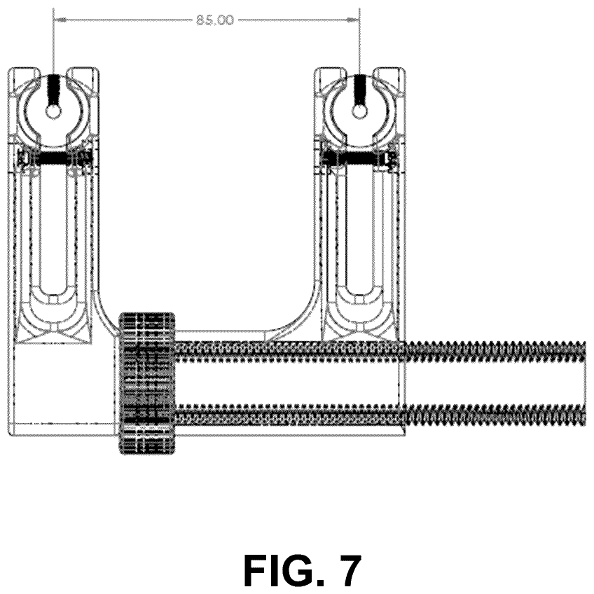

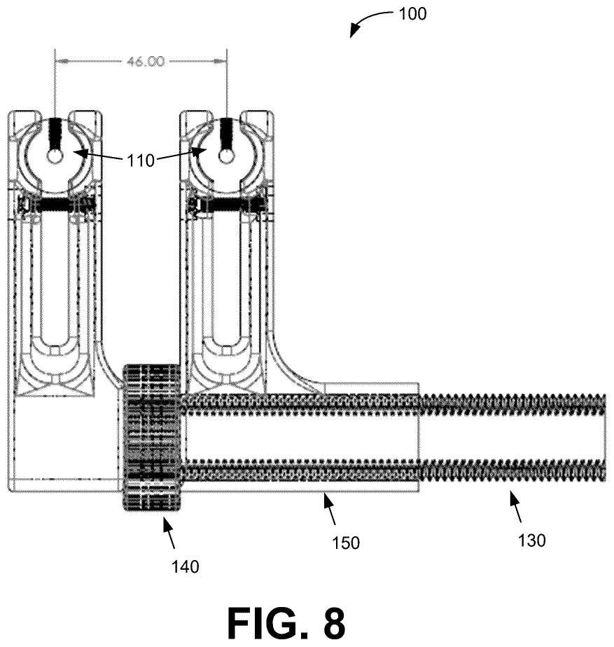

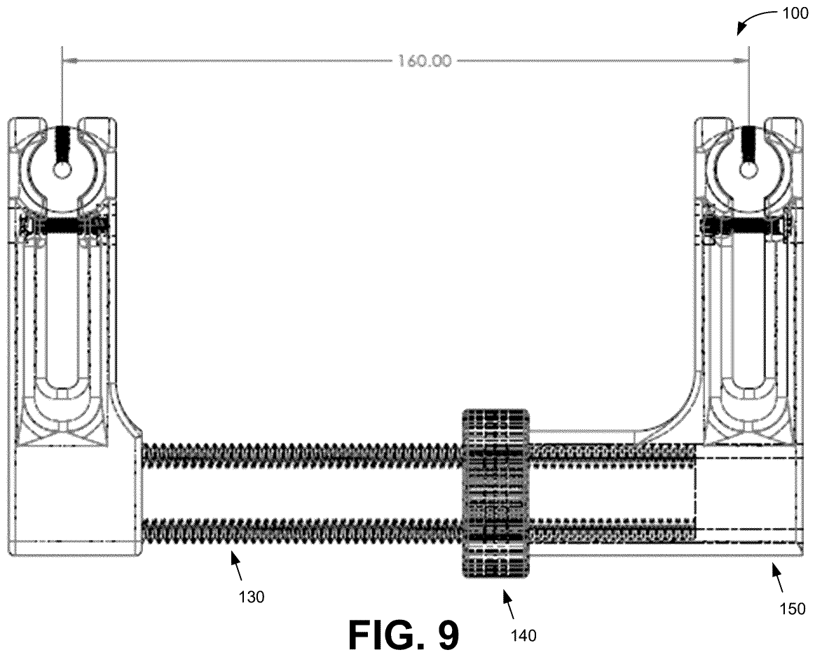

Next, as shown in FIG. 6, the spherical ball guide component 130 may have, but is not limited to only having, a 3.6 mm central hole and an integrated thread for the set screw 112 that allows the guide pin to be locked into the spherical ball guide 110. FIG. 7 shows the slider distractor arm 150 telescoped along the threaded distractor arm 130 in the maximally shortened position, with the distractor nut 140 advanced to the end of the threaded distractor arm 130. This position enables the spherical ball guides 110 (and the guide pins secured to the ball guides) to be placed 85 mm apart, in a non-limiting embodiment. By reversing the direction of the slider distractor arm 150, this distance can be decreased to 46 mm, as depicted in FIG. 8, in a non-limiting embodiment. With the slider distractor arm 150 in the forward orientation, the maximum distance between these spherical ball guides 110 reaches 160 mm as depicted in FIG. 9, in a non-limiting embodiment.

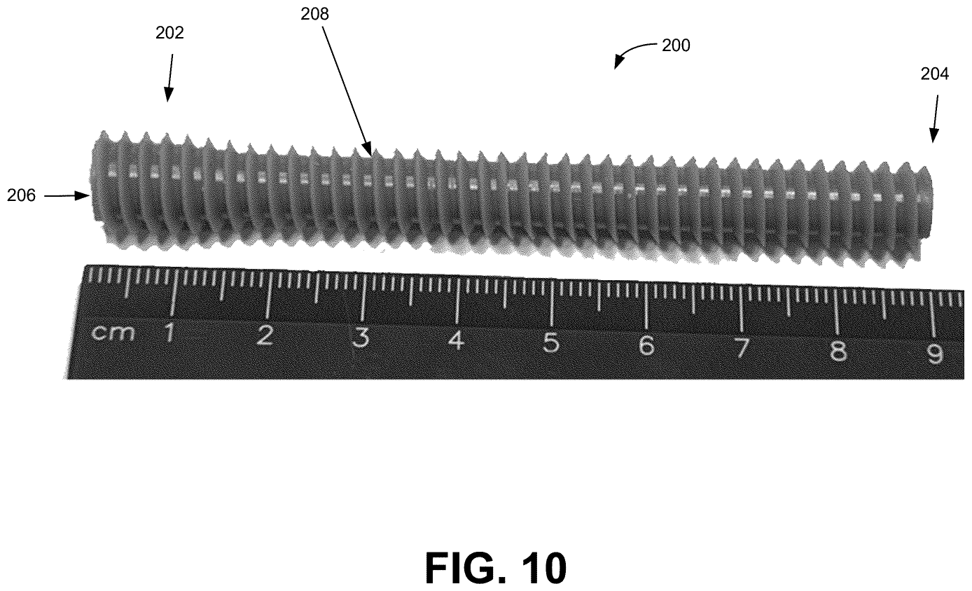

Referring now to FIG. 10, a 3D printed model of a novel distractor fastener 200 (e.g., a screw) is illustrated. In various embodiments, the novel distractor fastener 200 may be manufactured from various solid materials, such as hard resins, metals, polymers, etc. In various embodiments, the distractor fastener 200 comprises an elongated cylindrical body having a first end 202 and a second end 204, wherein an outer diameter tapers along the elongated cylindrical body from the first end to the second end, the outer diameter of the first end being equal to or greater than the outer diameter of the second end, and wherein a central channel 206 having an inner diameter extends axially through the elongated cylindrical body; and helical threads 208 traversing along the elongated cylindrical body from the first end to the second end, wherein the helical threads change a thread profile at a point along the elongated cylindrical body. In accordance with the present disclosure, a guide pin or wire can be inserted through the central channel 206 to guide positioning of the distractor fastener 200.

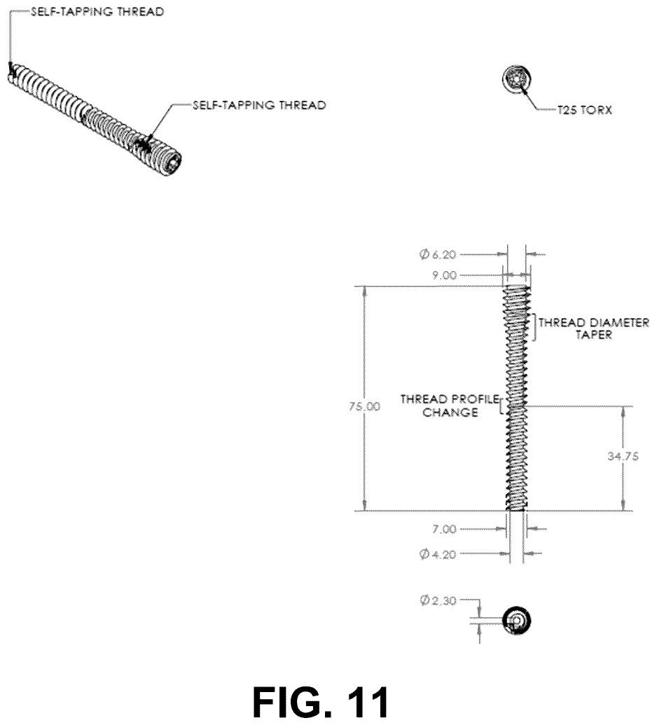

In various embodiments, the novel fastener 200 is a fully threaded screw having a tapered head, with the dimensions detailed in FIGS. 10-11 (e.g., length of approximately 75 mm to 90 mm), tapering from 9.0 mm down to 7.0 mm according to one non-limiting embodiment. Halfway along the length of the fastener 200, the thread direction changes (FIG. 11). This change in thread profile allows the fastener 200 to provide additional resistance against backing out of the bone as the bone shortens. This is important because the calcaneus bone will want to shorten again after fracture reduction due to where lengthening is performed, due to inherent forces affected on the bone from the surrounding soft tissues.

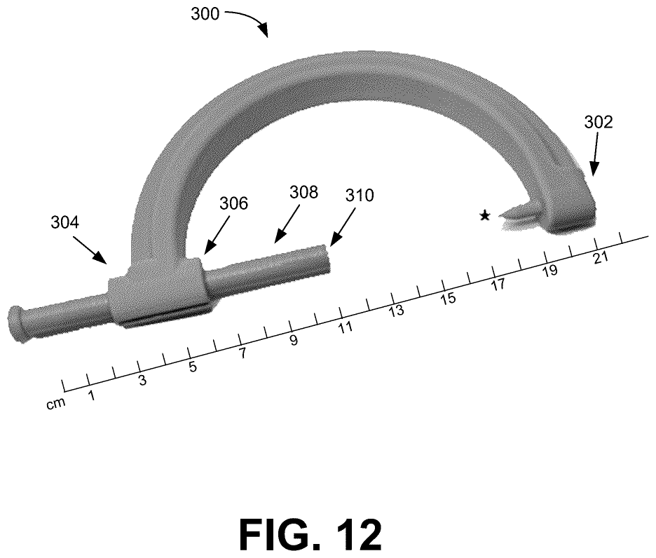

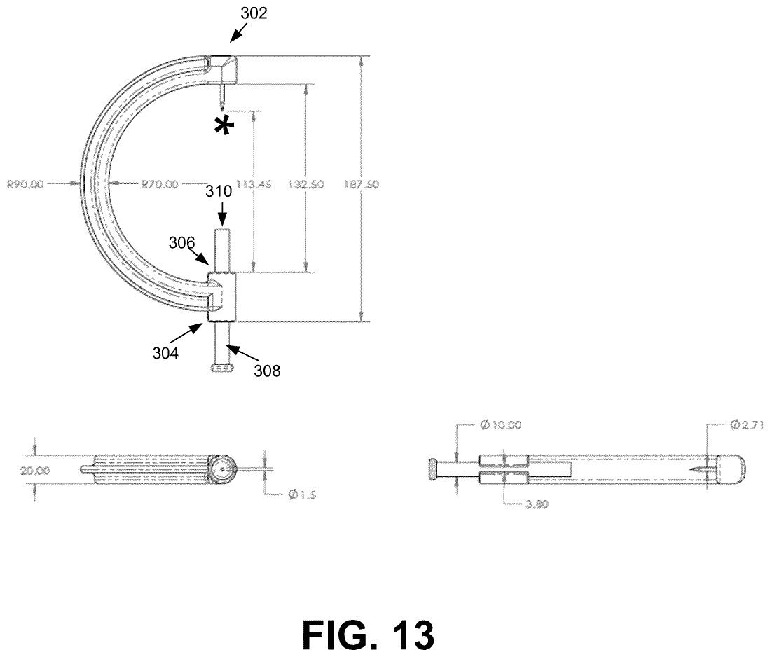

The largest fasteners 200 may be placed from posterior to anterior, and work to preserve calcaneal length and resist shortening. Commonly placed fasteners are one to two screws spanning from the lateral posterior facet into the sustentaculum of the calcaneus. This fastener trajectory can be challenging, and an exemplary drill targeting guide is designed to facilitate this fastener placement. Accordingly, FIG. 12 shows an image of a 3D printed model of a drill targeting guide 300 in accordance with various embodiments of the present disclosure. In turn, FIG. 13 is an illustration of an exemplary drill targeting guide 300 and its dimensions in accordance with various embodiments of the present disclosure, which may include a length of the drill targeting guide 300 being 187.50 mm, in a non-limiting embodiment. In various embodiments, the novel drill targeting guide 300 may be manufactured from various solid materials, such as hard resins, metals, polymers, etc.

In various embodiments, as shown in FIGS. 12-13, the drill targeting guide 300 comprises a semicircular body having a first end 302 and a second end 304, the first end having a target point (denoted by an asterisk (*) in FIG. 12) extending perpendicularly from the first end, the second end having a channel 306 extending perpendicularly through the second end, wherein the channel is configured to fit an elongated guide member 308 having an interior channel 310 for receiving a guide wire. In operations, the target point is placed externally on the target location, such as the sustentaculum, to help in guiding a guide wire through the elongated guide member towards the target point from the lateral side. The guide wire is then in place and able to be used for drilling and fastener placement along the desired trajectory.

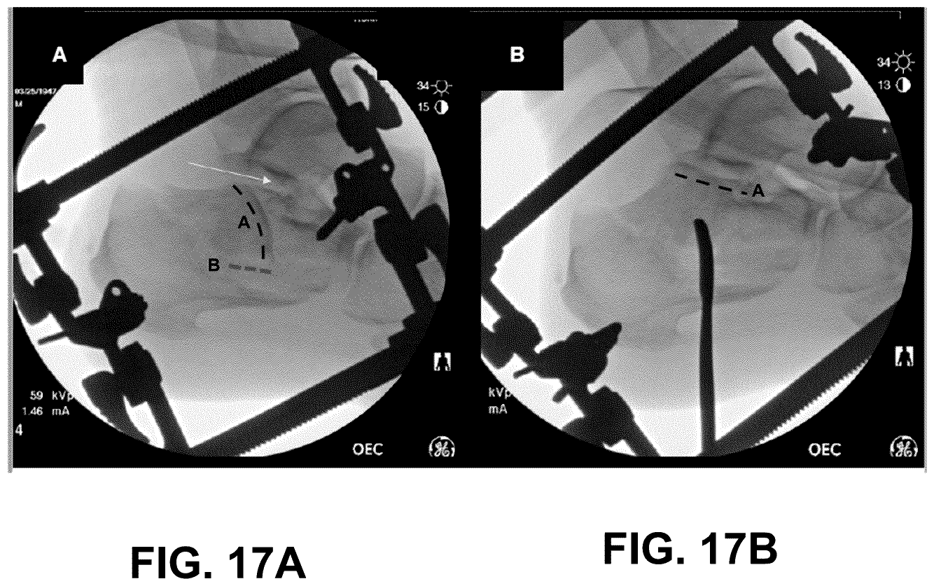

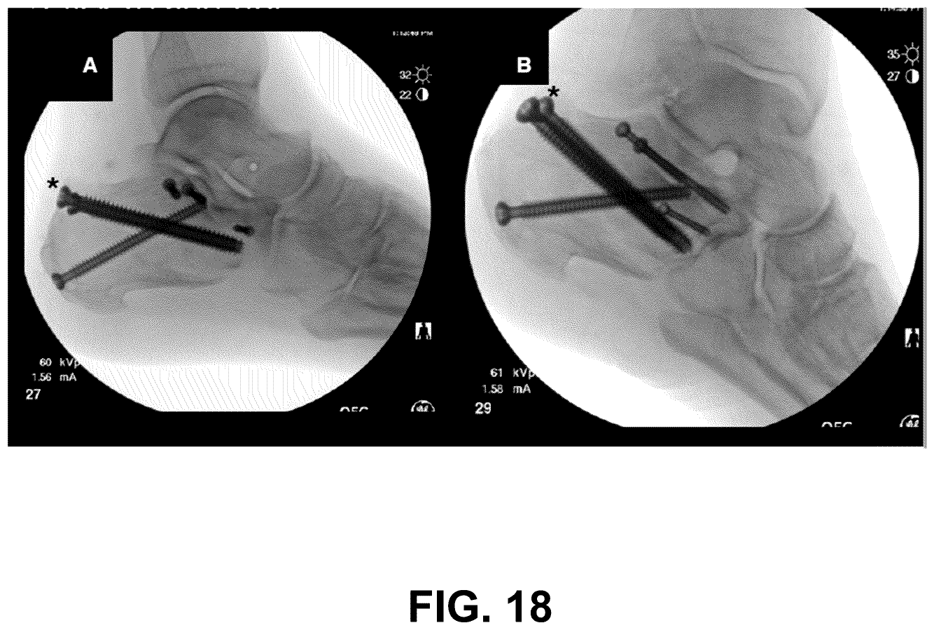

An exemplary method of the present disclosure includes various surgical steps as follows. As depicted in FIG. 14, a surgeon can place two metal guide pins (or wires) through the foot and ankle using an exemplary calcaneus distraction device 100, as disclosed in the present application. For example, one metal guide wire can be inserted through the talar neck of the central or antero-distal aspect (FIG. 14, PIN 1), perpendicular to the lateral profile of the talar dome on fluoroscopy. A second wire can then be placed in the inferior and posterior in the calcaneus, perpendicular to the varus deformity as visualized on an axial view under fluoroscopy (FIG. 16A). With the described calcaneus distractor device attached both medially and laterally, a distractive force applied between these guide pins (e.g., to spread the guide pins apart) helps to restore calcaneal length and height, and correct calcaneal varus (FIG. 15, FIG. 16A-B). With room created for a reduction of the posterior facet fragment (FIG. 17A, arrow), this fragment (orientation denoted by black dashed line FIG. 17B (denoted with an “A”)) can then be manipulated in a percutaneous fashion. This is done by making an incision beneath the osteochondral fragment (incision site denoted FIG. 17A, dashed line (denoted with a B)), and then inserting an elevator or similar tool. A sinus tarsi incision can be made if adequate reduction is not achieved. FIG. 17B shows significant change in orientation of the osteochondral fragment (FIG. 17A-B, black dashed line (denoted with an A)) after manipulation, which was made possible by the distractive forces from the present invention. The reduction of the calcaneus fragments can then be secured, with example images of fastener (e.g., screw) fixation detailed in FIG. 18A-B and FIG. 16C. The largest fasteners (e.g., screws) (FIG. 18A-18B, denoted with an *) are placed from posterior to anterior, and work to preserve calcaneal length and resist shortening. Commonly placed fasteners are one to two screws spanning from the lateral posterior facet into the sustentaculum of the calcaneus (FIG. 18A-18B). This screw trajectory can be challenging, and the disclosed drill targeting guide (FIG. 12, 13) is enabled to facilitate this screw placement. The sustentaculum (target) is palpable medially, and the target point (FIG. 12, *) is placed on this prominence to help guide wire placement. A guide wire is then placed through the opposite side of the drill targeting guide after the desired start point is located to allow for the guide wire to be in position for drilling and screw placement along the desired trajectory.

As discussed, various embodiments of the present disclosure include a calcaneus distraction device 100. In accordance with various embodiments, an exemplary calcaneus distraction device includes a threaded distractor arm 130 having an L-shape comprising a first elongated longitudinal body 131 (FIG. 1) extending between a first end 132 and a second end 134, and a second elongated longitudinal body 135 (FIG. 1) extending between the second end 134 and a third end 136, where the first elongated longitudinal body 131 and the second elongated longitudinal body 135 are connected at an approximately 90 degree angle to one another. The first end has at least a first ball socket 120 and the second elongated longitudinal body 135 has threads on at least two opposing surfaces of the second elongated longitudinal body 135. The third end of the second elongated longitudinal body 135 is configured to removably couple with a sliding distractor arm 150 of the calcaneus distraction device 100.

Accordingly, in various embodiments, the sliding distractor arm 150 has an L-shape comprising a third elongated longitudinal body 151 (FIG. 1) extending between a fourth end 152 and a fifth end 154 and a fourth elongated longitudinal body 155 (FIG. 1) extending between the fifth end 154 and a sliding end 156, where the third longitudinal body 151 and the fourth longitudinal body 155 are connected at an approximately 90 degree angle to one another. In various embodiments, the fourth end 152 has at least a second ball socket 120. In various embodiments, the fourth elongated longitudinal body 155 has a channel throughout its longitudinal axis configured to fit the third end 136 of the threaded distractor arm 130, wherein the third end of the threaded distractor arm 130 is removably coupled to the sliding distractor arm 150 by a distractor nut 140 that is configured to rotate along the threads of the threaded distractor arm 130 to displace the sliding distractor arm 150 along the longitudinal axis of the threaded distractor arm 130.

In various embodiments, the calcaneus distraction device 110 further includes a plurality of spherical ball guides 110 configured to rotate within the first or second ball sockets 120, the spherical ball guides 110 having a through channel 113 (FIG. 2) configured to fit a guide pin and an actuator channel 114 (FIG. 2) configured to fit a set screw 112 for locking the guide pin into a spherical ball guide 110 at a desired position, wherein the actuator channel 114 is perpendicular to the through channel 113.

In various embodiments, the threaded distractor arm and the sliding distractor arm comprise a radiolucent material.

In various embodiments, the first end 132 of the threaded distractor arm 130 further comprises a first fastener channel 124 adjacent to the first ball socket 110, the first fastener channel 124 extending perpendicularly through the first end and being configured to fit a first fixation fastener (e.g., a torx screw) 122.

In various embodiments, the fourth end 152 of the sliding distractor arm 150 further comprises a second fastener channel 124 adjacent to the second ball socket 110, the second fastener channel 124 extending perpendicularly through the fourth end and being configured to fit a second fixation fastener (e.g., a torx screw) 122.

It should be emphasized that the above-described embodiments are merely possible examples of implementations, merely set forth for a clear understanding of the principles of the present disclosure. Many variations and modifications may be made to the above-described embodiment(s) without departing substantially from the principles of the present disclosure. All such modifications and variations are intended to be included herein within the scope of this disclosure.

Therefore, at least the following is claimed:

1. A calcaneus distraction system comprising:
a calcaneus distraction device having:
a threaded distractor arm having an L-shape comprising a first elongated longitudinal body extending between a first end and a second end, and a second elongated longitudinal body extending between the second end and a third end, wherein the first elongated longitudinal body and the second elongated longitudinal body are connected at an approximately 90 degree angle to one another,
the first end having at least a first ball socket and the second elongated longitudinal body having threads on at least two opposing surfaces of the second elongated longitudinal body, the third end being configured to removably couple with a sliding distractor arm;
the sliding distractor arm having a third elongated longitudinal body extending between a fourth end and a fifth end, and a fourth elongated longitudinal body extending between the fifth end and a sliding end, wherein the third elongated longitudinal body and the fourth elongated longitudinal body are connected at an approximately 90 degree angle to one another,
the fourth end having at least a second ball socket and the fourth elongated longitudinal body having a channel throughout its longitudinal axis configured to fit the third end of the threaded distractor arm, wherein the third end of the threaded distractor arm is removably coupled to the sliding distractor arm by a distractor nut that is configured to rotate along the threads of the threaded distractor arm to displace the sliding distractor arm along the longitudinal axis of the threaded distractor arm; and
a plurality of spherical ball guides configured to rotate within the first or second ball sockets, the spherical ball guides having a through channel configured to fit a guide pin.

2. The calcaneus distraction system of claim 1, wherein the spherical ball guides comprise and an actuator channel configured to fit a set screw for locking the guide pin into a spherical ball guide at a desired position, wherein the actuator channel is perpendicular to the through channel.

3. The calcaneus distraction system of claim 1, wherein the threaded distractor arm and the sliding distractor arm comprise a radiolucent material.

4. The calcaneus distraction system of claim 1, wherein the first end further comprises a first fastener channel adjacent to the first ball socket, the first fastener channel extending perpendicularly through the first end and being configured to fit a first fixation fastener.

5. The calcaneus distraction system of claim 4, wherein the fourth end further comprises a second fastener channel adjacent to the second ball socket, the second fastener channel extending perpendicularly through the fourth end and being configured to fit a second fixation fastener.

6. The calcaneus distraction system of claim 1, further comprising:
a distractor fastener having
an elongated cylindrical body having a sixth end and a seventh end, wherein an outer diameter tapers along the elongated cylindrical body from the sixth end to the seventh end, the outer diameter of the sixth end being equal to or greater than the outer diameter of the seventh end, and wherein a channel having an inner diameter extends axially through the elongated cylindrical body; and
helical threads traversing along the elongated cylindrical body from the sixth end to the seventh end, wherein the helical threads change a thread profile at a point along the elongated cylindrical body.

7. The calcaneus distraction system of claim 1, further comprising:
a drill targeting guide comprising a semicircular body having a sixth end and a seventh end, the sixth end having a target point extending perpendicularly from the sixth end, the seventh end having a channel extending perpendicularly through the seventh end, wherein the channel is configured to fit a guide wire.

8. The calcaneus distraction system of claim 7, further comprising:
a distractor fastener having
an elongated cylindrical body having an eighth end and a ninth end, wherein an outer diameter tapers along the elongated cylindrical body from the eighth end to the ninth end, the outer diameter of the eighth end being equal to or greater than the outer diameter of the ninth end, and wherein a channel having an inner diameter extends axially through the elongated cylindrical body that is configured to receive the guide wire after being positioning using the drill targeting guide; and
helical threads traversing along the elongated cylindrical body from the eighth end to the ninth end, wherein the helical threads change a thread profile at a point along the elongated cylindrical body.

9. A method of calcaneus distraction comprising:
placing one or more guide pins through an ankle;
attaching one or more calcaneus distraction devices to the one or more guide pins;
distracting the one or more guide pins using the one or more calcaneus distraction devices; and
securing reduction of calcaneus fragments through fixation of a distractor fastener, wherein the fixation of the distractor fastener is facilitated by a drill targeting guide.

10. The method of claim 9, wherein the one or more one or more calcaneus distraction devices comprise:
a threaded distractor arm having an L-shape comprising a first elongated longitudinal body extending between a first end and a second end, and a second elongated longitudinal body extending between the second end and a third end, wherein the first elongated longitudinal body and the second elongated longitudinal body are connected at an approximately 90 degree angle to one another,
the first end having at least a first ball socket and the second elongated longitudinal body having threads on at least two opposing surfaces of the second elongated longitudinal body, the third end being configured to removably couple with a sliding distractor arm;
the sliding distractor arm having a third elongated longitudinal body extending between a fourth end and a fifth end, and a fourth elongated longitudinal body extending between the fifth end and a sliding end, wherein the third elongated longitudinal body and the fourth elongated longitudinal body are connected at an approximately 90 degree angle to one another,
the fourth end having at least a second ball socket and the fourth elongated longitudinal body having a channel throughout its longitudinal axis configured to fit the third end of the threaded distractor arm, wherein the third end of the threaded distractor arm is removably coupled to the sliding distractor arm by a distractor nut that is configured to rotate along the threads of the threaded distractor arm to displace the sliding distractor arm along the longitudinal axis of the threaded distractor arm; and
a plurality of spherical ball guides configured to rotate within the first or second ball sockets, the spherical ball guides having a through channel configured to fit a guide pin.

11. The method of claim 10, wherein the spherical ball guides comprise an actuator channel configured to fit a set screw for locking the guide pin into a spherical ball guide at a desired position, wherein the actuator channel is perpendicular to the through channel.

12. The method of claim 9, wherein the distractor fastener comprises:
an elongated cylindrical body having a fifth end and a sixth end, wherein an outer diameter tapers along the elongated cylindrical body from the fifth end to the sixth end, the outer diameter of the fifth end being equal to or greater than the outer diameter of the sixth end, and wherein a channel having an inner diameter extends axially through the elongated cylindrical body that is configured to receive a guide wire; and
helical threads traversing along the elongated cylindrical body from the fifth end to the sixth end, wherein the helical threads change a thread profile at a point along the elongated cylindrical body.

13. The method of claim 9, wherein the drill targeting guide comprises a semicircular body having a fifth end and a sixth end, the fifth end having a target point extending perpendicularly from the fifth end, the sixth end having a channel extending perpendicularly through the sixth end, wherein the channel is configured to fit a guide wire.

14. The method of claim 13, wherein the distractor fastener comprises:
an elongated cylindrical body having a seventh end and an eighth end, wherein an outer diameter tapers along the elongated cylindrical body from the seventh end to the eighth end, and wherein a channel having an inner diameter extends axially through the elongated cylindrical body that is configured to receive the guide wire after being positioned using the drill targeting guide; and
helical threads traversing along the elongated cylindrical body from the seventh end to the eighth end, wherein the helical threads change a thread profile at a point along the elongated cylindrical body.

15. The method of claim 14, wherein the outer diameter of the seventh end is equal to or greater than the outer diameter of the eighth end.
